(19) United States
(12) Patent Application Publication
(10) Pub. No.: US 2026/0275282 A1
(43) Pub. Date: Sep. 17, 2026

(54) CELLS BEING HIGHLY ENRICHED IN SASP FACTORS, AND COMPOSITION COMPRISING SAID CELLS

(71) Applicant: EVOMEDIS GMBH, Graz (AT)

(72) Inventor: Martin Funk, Landsberg Am Lech (DE)

(73) Assignee: EVOMEDIS GMBH, Graz (AT)

(21) Appl. No.: 19/130,027

(22) PCT Filed: Nov. 16, 2022
(86) PCT No.: PCT/EP2022/082124

§ 371 Date: May 14, 2025

(51) Int. Cl. C12N 5/071; A61K 35/36; A61P 17/02

(52) U.S. Cl. CPC C12N 5/0629; A61K 35/36; A61P 17/02; C12N 2501/727

(57) ABSTRACT

The present invention refers to a cell having the capacity of producing at least one SASP factor, obtainable or obtained by treating the cell with an amount of an exogenous secretion inhibitor that is sufficient to inhibit the secretion of the at least one SASP-factor. The present invention further refers to a cell that is intracellularly enriched in at least one SASP factor selected from a defined group of SASP factors, to a composition comprising said cells, as well as to this cell and composition for use in a therapeutic and/or prophylactic treatment, and also for use in tissue regeneration. The present invention also refers to a method for producing a cell that is intracellularly enriched in at least one endogenously produced SASP factor.

### FIELD OF THE INVENTION

The present invention refers to a cell having the capacity of producing at least one SASP factor, obtainable or obtained by treating the cell with an amount of an exogenous secretion inhibitor that is sufficient to inhibit the secretion of the at least one SASP-factor. The present invention further refers to a cell that is intracellularly enriched in at least one SASP factor selected from a defined group of SASP factors, to a composition comprising said cells, as well as to this cell and composition for use in a therapeutic and/or prophylactic treatment, and also for use in tissue regeneration. The present invention also refers to a method for producing a cell that is intracellularly enriched in at least one endogenously produced SASP factor.

### DESCRIPTION OF THE BACKGROUND ART

Biologically active products comprising cells, for example autologous or allogeneic keratinocytes, either alone or in combination, are well established the manufacture of products for treating wounds, particularly burns or ulcers. While in former times allogeneic keratinocytes were used for skin replacement (see for example [1]), it was found that the success of the treatment is not due to the keratinocytes growing into the wound and “replacing” cells. Instead, transplanted keratinocytes solely remain at the wound for a certain period of time and then vanish or are removed, but stimulate the body's own healing process by re-epithelialization due to a complex network of secreted factors (see for example [2]).

Notably, keratinocytes' products known in the art mainly focus on proliferating and non-senescent cells.

However, such keratinocyte products are either complex to gain, cumbersome to applicate, intricate to store and/or do not provide a cocktail of biologically active factors that are particularly suitable for efficiently treating wounds. Therefore, products are currently developed that are composed of freeze dried [3, 4] primary and senescent keratinocytes for the treatment of hard to heal burn wounds and chronic wounds.

In this regard, e.g., European patent EP 3 325 025 B1 discloses the use of senescent cells for tissue regeneration.

During the process of cell amplification, which is required to produce senescent keratinocytes, senescence occurs mainly at later cell passages and can be blocked. After amplification, senescence of the cells can be induced by different stimuli, e.g., as described in EP 3 325 025 B1, or as summarized for example by Petrova et al. [5]. This leads to the production and secretion of so-called SASP (senescence-associated secretory phenotype) factors that can stimulate wound healing [6, 7].

So far, the cell therapeutic products in the prior art are composed of freeze dried or non-freeze dried, and non-proliferating cells. Therefore, the majority of SASP factors produced by the cells is delivered from the intracellular pool of the cells: It is the current understanding that in the first days of wound healing, the cells that are applied to the wound undergo decomposition, thereby releasing the SASP factors produced by the cells.

It is an object of the present invention to provide an improved status of cells, which can beneficially contribute to useful therapeutic and prophylactic treatments such as tissue regeneration and specifically e.g. to wound healing. The cell whose therapeutic status is to be improved shall have the capacity of producing at least one SASP factor. The cell whose therapeutic status is to be improved may generate or may already contain said at least one SASP factor, that is the cell may already be endogenously enriched or loaded with, preferably highly enriched or loaded, with said at least one SASP factor. Accordingly, the cells may generate or already contain amounts of SASP factors which are substantially large and then, by way of the treatment according to the present invention, are capable of maintaining an enriched or loaded and preferably a highly enriched or loaded amount of SASP factors, which in turn subsequently, e.g. upon application of said cells or compositions derived from said cells to a wound and released to the wound, can display its useful therapeutic effects.

It is further an object of the present invention to provide a method for producing such cells, and to provide an improved composition which can be used in therapeutic and/or prophylactic treatment.

These as well as further objects, which will become apparent from the following description of the present invention, are attained by the subject matter of the independent claims. Some of the preferred embodiments of the present invention are defined by the subject matter of the dependent claims.

### SUMMARY OF THE INVENTION

Various aspects, advantageous features and preferred embodiments of the present invention as summarized in the following items, respectively alone or in combination, contribute to solving the object of the invention.
1. Cell having the capacity of producing at least one SASP factor, obtainable or obtained by treating (modifying) the cell with an amount of an exogenous secretion inhibitor that is sufficient to at least reduce, preferably inhibit, the secretion of the at least one SASP-factor.

The term “reduction” defines that the secretion of the at least one SASP factor is reduced by 10% or more, i.e. the remaining secretion of the SASP factor is then 90% or less; preferably the secretion of the at least one SASP factor is reduced by 15% or more, preferably by 20% or more, more preferably by 30% or 40% or more, even more preferably by 50% or more, and particularly preferred by 60% or more. In a particularly preferred embodiment, the secretion of the at least one SASP factor is 70% or more, and in a most preferred embodiment, the secretion is inhibited.

The (degree of) reduction can be determined by comparing the amount of the respective SASP factor(s) in the supernatant without treatment and with treatment with the exogenous secretion inhibitor, e.g., by using an assay as disclosed elsewhere herein.
2. Cell according to item 1, wherein the cell, as a result of the treatment with the exogenous secretion inhibitor, exhibits the following capacities (i) to (iii):
(i) a cellular structure or cellular function that is different to the cellular structure or cellular function without the respective treatment; and additionally
(ii) at least reduced, preferably inhibited, ability to secrete the at least one SASP-factor; and/or
(iii) is intracellularly enriched in the at least one SASP-factor, preferably wherein the cell, as a result of the treatment with the exogenous secretion inhibitor, exhibits capacity (i), and additionally (iii), more preferably the cell exhibits capacity (i), (ii), and (iii).

In other words, in a preferred embodiment, the treated cell, as a result of the treatment, exhibits the capacities (features) (i), (ii), and (iii). It is however also possible that the treated cell, as a result of the treatment, exhibits capacities (i) and (iii) only.
3. Cell according to item 1 or 2, wherein the at least one SASP factor is selected from the group consisting of IL-1α, IL-1β, IL-6, IL-7, IL-8, IL-10, IL-13, IL-15, IL-18, MCP1, MCP2, MCP4, MIF, MIP-1a, MIP-3a, HCC-4, Eotaxin-3, TECK, ENA-78, I-309, I-TAC, GROα, GROβ, GROγ, VEGF, EGF, HGF, HBEGF, FGF, such as FGF acidic, bFGF, KGF, NGF, Amphiregulin, Angiogenin, APOJ, CAV1, OSTEO, Epiregulin, Heregulin, SCF, SDF-1 alpha, PIGF, IGF-1, IGFBP-1, -2, -3, -4, -5, -6, -7, GM-CSF, PDGF-BB, TGF-α, TGF-β1, TGF-β2, TGF-β3, TRAIL-R3, Fas, OPG, SGP130, EGF-R uPAR, sTNFRI, sTNFRIII, MMP1, MMP2, MMP3, MMP7, MMP9, MMP10, MMP12, MMP13, MMP14, TIMP1, TIMP2, PAI1, PAI2, Park7 DJ-1, uPA/Urokinase, SLPI, SPP1, Syndecan 1, -4, Tenascin C, Endothelin, Collagens, Fibronectins, Laminins, ICAM1, and ICAM3.

In a further embodiment, the cell is intracellularly (endogenously) enriched in at least one or more, preferably all of the following SASP factors: IL-1 alpha, IL-6, IL-8, TGF-α, GROα, VEGF, EGF, FGF acidic, MMP-7, and TIMP1.
4. Cell according to item 2 or 3, wherein the cellular structure or cellular function that is different to the respective cellular structure or cellular function without the respective treatment is one or more selected from the group consisting of:
(i-i) function of protein insertion into the endoplasmic reticulum (ER);
(i-ii) function of protein maturation;
(i-iii) function of protein trafficking and/or vesicle formation;
(i-iv) function of microtubules; and
(i-v) function of kinesin;
(i-vi) function of autophagy.
5. Cell according to any of items 1 to 4, wherein the exogenous secretion inhibitor is selected from the group consisting of any of (i) to (vi):
(i) Group I: exogenous secretion inhibitor that affects the function of import into the endoplasmic reticulum (ER);
(ii) Group II: exogenous secretion inhibitor that affects the function of protein maturation;
(iii) Group III: exogenous secretion inhibitor that affects the function of protein trafficking and/or vesicle formation;
(iv) Group IV: exogenous secretion inhibitor that affects the function of microtubules;
(v) Group V: exogenous secretion inhibitor that affects the function of kinesin; and
(vi) Group VI: exogenous secretion inhibitor that affects the function of autophagy.
6. Cell according to any of items 1 to 5, wherein
(i) the exogenous secretion inhibitor from Group I is selected from the group of compounds that affect, preferably inhibit, endoplasmic reticulum (ER) import (group 1), e.g. Mycolactone; Cotransisns, e.g. HUN-729; Apratoxins, e.g. Apratoxin A, Decatransin, Coibamide A, Ipomeassin-F, Spiperone;
(ii) the exogenous secretion inhibitor from Group II is selected from the group of compounds that affect, preferably inhibit, maturation of proteins (group II), e.g. Sesquiterpene lactones, e.g. thapsigargin; Basiliolides, e.g. Basiliolide; Inhibitors of protein glycosylation, e.g. Tunicamycin; Compounds blocking the eukaryotic signal peptidase, e.g. Cavinafungin;
(iii) the exogenous secretion inhibitor from Group III is selected from the group of compounds that affect, preferably inhibit, protein trafficking and/or vesicle formation (group III), e.g. Brefeldin A; and vesicle formation inhibitors, e.g. Calpastatin, Calpeptin, Calpain inhibitor, Manumycin A, GW4689, Y27632;
(iv) the exogenous secretion inhibitor from Group IV is selected from the group of compounds that affect, preferably inhibit, tubulin (group IV), such as
   microtubule-destabilizing agents with vinca-domain,
   e.g. Vinblastin, Vincristine, Vindesine, Vinorelbine, Eribulin, E7974, Tubulsin A, Cryptophycin 52, Halichondrins such as E7389, Dolastatins such as TZT-1027, Hemiasterlins such as HTI-286;
   microtubule-destabilizing agents with colchicine domain,
   e.g. Colchicine, BPROLO75, Indublin, Combretastatins e.g. AVE8062A, CA-1-P, CA-4-P, N-acetylcolchicinol-O-phosphate, ZD6126; 2-Methoxyestradiol; Methoxybenzene-sulphonamide such as ABT-751, E7010; 2-methoxy-5-(2,3,4-trimethoxyphenyl) 2,4,6-cycloheptatrien-1-one;
   microtubule-stabilizing agents with taxane domain,
   e.g. taxanes such as Paclitaxel (Taxol), TL00139 and analogues thereof, docetaxel (Taxotere), Epothilones such as BMS-247550, Epothilones B and D, Discodermolide;
   microtubule-stabilizing agents with laulimalide domain,
   e.g. Epothilones, such as Desoxyepothilone B;
   microtubule-assembly inhibitors,
   e.g. Astemizole, Parbendazole;
(v) the exogenous secretion inhibitor from Group V is selected from the group of compounds that affect, preferably inhibit, the function of kinesin, e.g. Ispinesib (SB715992); SB743921 (hydrochloride); GSK923295 (an allosteric inhibitor of centromere-associated protein-E (CENP-E) kinesin motor ATPase activity); GW406108X (a Kif15 inhibitor); Dimethylenastron; ARQ 621 (inhibitor of Eg5, a microtubule-based ATPase motor protein involved in cell division);
(vi) the exogenous secretion inhibitor from Group VI is selected from the group of compounds that affect autophagy, such as compounds that induce autophagy, for example the autophagy inducers listed in Table 1; and compounds that inhibit autophagy, for example the autophagy inhibitors listed in Table 1.
7. Cell according to any of the preceding items, wherein the cell is selected from the group consisting of keratinocytes, fibroblasts, epithelial cells, melanocytes, endothelial cells, corneal epithelial cells, pericytes, monocytes, lymphocytes, thrombocytes, mast cells, adipocytes, muscle cells, neurons, osteocytes, osteoblasts, chondrocytes, mesenchymal stem cells, and adult or embryonic stem cells, preferably the cells are selected from the group consisting of epithelial cells, corneal epithelial cells, keratinocytes, fibroblasts, melanocytes, endothelial cells, pericytes, monocytes, lymphocytes, thrombocytes, mast cells, adipocytes, mesenchymal stem cells, and/or adult or embryonic stem cells, more preferably, the cell is a skin-associated cell, particularly the cell is selected from the group consisting of keratinocytes, fibroblasts, adipocytes and endothelial cells.
8. Cell according to any of the preceding items, wherein the cell is of a senescent cell type.
9. Cell according to item 8, wherein the senescent cell type derives from the cells as defined in claim 7.
10. Cell being intracellularly enriched in at least one SASP-factor selected from the group consisting of IL-1 alpha, IL-6, IL8, TGF-alpha, GRO alpha, VEGF, EGF, FGF acid, MMP7, and TIMP1, wherein the intracellular concentration, calculated as pg/mg protein extract, of said one or more SASP factor, if present, is defined as follows:

| | intracellular | | | |
|---|---|---|---|---|
| | (pg/mg protein | | | even more |
| | extract) | preferred | more preferred | preferred |
| IL-1 alpha | more than 80 | more than 80 to | 1.000 to 15.000 | 2.000 to 10.000 |
| | | 20.000 | | |
| IL-6 | more than 2 | more than 2 to | 20 to 150 | 50 to 100 |
| | | 200 | | |
| TGF- alpha | more than 20 | more than 20 to | 100 to 2.000 | 150 to 1.000 |
| | | 2.500 | | |
| IL8 | more than 20 | more than 20 to | 50-1.000 | 50-250 |
| | | 1000 | | |
| GRO alpha | more than 20 | more than 20 to | 50-1.000 | 50-250 |
| | | 1000 | | |
| VEGF | more than 20 | more than 20 to | 50-1.000 | 50-250 |
| | | 1000 | | |
| EGF | more than 10 | more than 10 to | 100 to 1.500 | 200 to 1.000 |
| | | 2.000 | | |
| FGF acidic | more than 100 | more than 100 | 1.000-15.000 | 2.000-10.000 |
| | | to 20.000 | | |
| MMP7 | more than 200 | more than 200 | 2.000-20.000 | more than 10.000 |
| | | to 20.000 | | to 20.000 |
| TIMP1 | more than 200 | more than 200 | 2.000-20.000 | more than 10.000 |
| | | to 20.000 | | to 20.000 |

The cell can also be a cell that is intracellularly enriched in at least one SASP-factor selected from the group consisting of IL-1 alpha, IL-6, TGF-alpha, EGF, FGF acidic, MMP7, and TIMP1, wherein the respective intracellular concentration, calculated as pg/mg protein extract, of said one or more SASP factor, if present, is as defined in the table above.
11. Cell according to any of items 1 to 10, wherein the cell is a frozen, spray-dried or freeze-dried cell, or the cell is present in or transferred into a frozen, dried, spray-dried or freeze-dried composition.
12. Composition comprising the cell as defined in any of items 1 to 11.
13. Composition according to item 12, wherein the cell is a frozen, spray-dried or freeze-dried cell.
14. Composition according to item 12 or 13, wherein the composition comprises a matrix substance.
15. Composition according to any of items 12 to 14, wherein the matrix substance comprises an excipient selected from sulfoxides and amides, e.g. DMSO; alcohols and derivatives, e. g. glycerol, glycol, ethylene glycol; sugars, e.g. glucose, galactose, lactose, sucrose, trehalose, dextran, erythriol, mannitol, inositol, sorbitol; amino acids, e.g. arginine, aspartic acid, glycine, leucine, phenylalanine, thereonine, trileucine, histidine; cellulose derivatives, e.g. hydroxy propyl methyl cellulose starch, starch derivatives, e.g., hydroxyethylstarch; organic osmolites e.g. ectoine, 5-hydroxyectoine, taurine, TMAO, gylcine betaine, GPC choline-O-sulphate, ß-DMSP; antioxidants, e.g. ascorbic acid, glutathione, α-tocopherol, arbutin, (−)-epigallocatechingallate, chelating agents, e.g. EGTA, EDTA, proteins, e.g. serum albumin, peptones; other polymeric substances, e.g. polyethylene glycol, polyvinyl pyrrolidone, ficoll, poloxameres, carbopol, chitosan.
16. Composition according to any of items 12 to 15, wherein the composition further comprises, or is applied to, a carrier selected from the group consisting of a wound dressing, a paste, a spray or an ointment, preferably the carrier is a wound dressing.
17. Composition according to item 16, wherein the wound dressing is jelly gauze, preferably petroleum jelly gauze, and/or wherein the carrier comprises or contains alginates, collagen, polyuratane foames, hydrocolloids, hydrofibers, hydrogels, hyaluronic acid, cellulose, an acellular animal or human derived product from skin or intestine like Oasis or biosynthetic materials like Laserskin or Suprathel; preferably, the carrier comprises or contains cellulose.
18. Composition according to any of items 12 to 17, wherein the composition additionally contains a functional substance selected from the group of: antimicrobial agents, antidiabetic agents, mRNA or miRNA antagonists against mRNAs or miRNAs over expressed in chronic, non-healing wounds, growth factors like for example PDGF, FGF-2, VEGF-A, -B, -C, -D, GM-CSF, SDF-1alpha, IL1-beta or peptides derived from these growth factors, inhibitors of enzymes being involved in cortisol synthesis, particularly CYP11B1 overexpressed in chronic wounds as well as of Prolyl-4-hydroxylase, elastase, GSK3ß phosphorylation and Cx43.
19. Composition according to item 18, wherein the antimicrobial agent is an antibiotic and/or an antifungal agent, preferably wherein the antibiotic is selected from the group consisting of: cephalosporines, particularly Cefazolin, Cefoxitin, Cefofetan; macrolides, particularly erythromycin; sulphonamides particularly Mafenide; penicillins, chlorhexidine, silver sulfadiazine, silver nitrate and silver derived formulations, and/or wherein the antidiabetic agent is a DDP-4 inhibitor.
20. Cell as defined in any of items 1 to 11, or composition as defined in any of items 12 to 19, for use in a therapeutic and/or prophylactic treatment.
21. Cell as defined in any of items 1 to 11, or composition as defined in any of items 12 to 19, for use in tissue regeneration.
22. Cell as defined in any of items 1 to 11, or composition as defined in any of items 12 to 19, for use in treatment of wounds.
23. Cell for use as defined in item 22, or composition for use as defined in item 22, wherein the wounds are acute or chronic wounds.
24. Cell for use as defined in item 22 or 23, or composition for use as defined in item 22 or 23, wherein the wounds are wounds of the skin, preferably wherein the wounds were treated by debridement before treatment with the composition and/or wherein the wounds were generated by split skin grafting and/or plastic surgery.
25. Cell for use as defined in any of items 22 to 24, or composition for use as defined in any of items 22 to 24, wherein the wounds are burns, particularly a burn selected from the group consisting of: superficial and/or deep partial thickness burn, second or third degree burn, sun burn; and/or wherein the wound is a chronic wound healing disorder selected from the group consisting of ulcers, pressure sores, diabetic foot syndrome.
26. Cell for use as defined in item 25, or composition for use as defined in item 25, wherein the ulcer is selected from the group consisting of ischemic, arterial, venous, neurotrophic, vasculitis, hypertensive and Pyoderma Gangrenosum, decubitus ulcers.
27. Cell for use as defined in any of items 22 to 25, or composition for use as defined in any of items 22 to 25, wherein the wound is selected from the group consisting of abrasions, any trauma, radiotherapy lesions, all types of skin loss.
28. Cell as defined in any of items 1 to 12, or composition as defined in any of items 13 to 19, for use in treatment of an inflammatory condition of skin and subcutaneous tissue, wherein the inflammatory condition is preferably selected from the group consisting of: Erythematosquamous dermatosis, Atopic dermatitis and related conditions, Contact dermatitis and other eczema, Dermatitis due to substances taken internally, Bullous dermatoses, Erythematous conditions, Psoriasis and similar disorders, Lichen, Pruritus and related conditions, and other diseases of skin and subcutaneous tissue like Actinic keratosis, Acne, Alopecia and other diseases of skin appendages.
29. Cell modified by treatment with a suitable amount of an exogenous secretion inhibitor, or composition comprising said modified cell, for use in a therapeutic and/or prophylactic treatment as defined in any of items 20 to 28.
30. Method for producing a cell that is intracellularly (endogenously) enriched in at least one endogenously produced SASP factor, comprising the following steps:
(a) providing a cell that is capable of producing one or more SASP factors;
(b) incubating the cell of (i) under suitable conditions with an exogenous secretion inhibitor in an amount that is sufficient to reduce, preferably inhibit, the secretion of the at least one SASP-factor;
(c) obtaining the cell of (ii) that is intracellularly enriched in at least one endogenously produced SASP factor.
31. Method according to item 30, wherein the obtained cell in step (c), as a result of the treatment with the exogenous secretion inhibitor,
(i) exhibits a cellular structure or cellular function that is different to the cellular structure or cellular function without the respective treatment;
and
(ii) exhibits reduced ability to secrete the at least one SASP-factor; and/or
(iii) is intracellularly enriched in the at least one SASP-factor.

Preferably, the obtained cell, as a result of the treatment with the exogenous secretion inhibitor, exhibits capacity (i), and additionally (iii), more preferably, the obtained cell exhibits capacity (i), (ii) and (iii).

### BRIEF DESCRIPTION OF THE DRAWINGS

FIG. 1 shows the principle steps and a selection of inhibitors of cellular protein secretion. Particularly, FIG. 1 depicts the secretory pathway of proteins towards the extracellular space and areas of inhibition promoted by different compounds. FIG. 1 is adapted from [8].

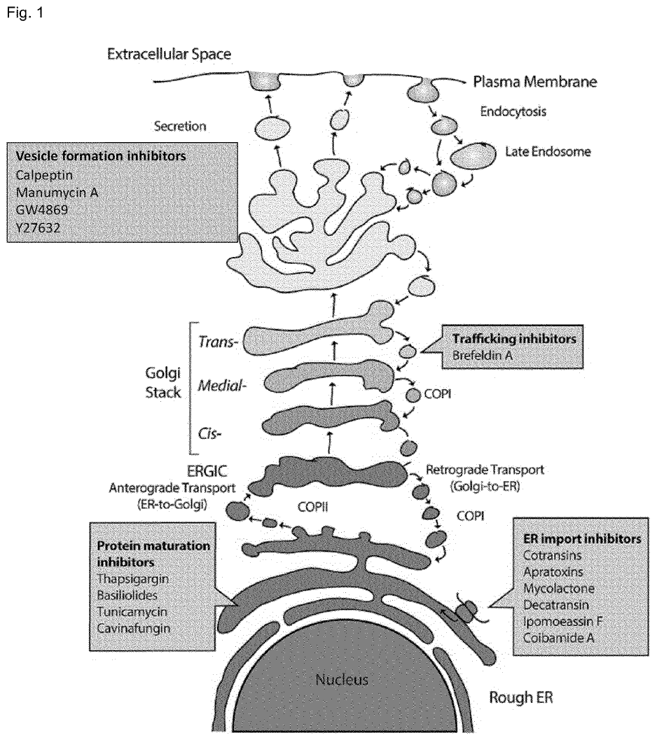

FIG. 2 shows the various cellular structures and cellular functions that can be affected by secretion inhibitors. It is a schematic figure of the protein expression in a hypothetical eukaryotic cell and its trafficking process until secretion. The figure contains the nuclei, the endoplasmic reticulum, the Golgi apparatus, and a microtubule network. In each of the presented steps, an inhibitor compound group (I to V; listed elsewhere herein) is highlighted. Figure was drawn using pictures from Servier Medical Art (https://smart.servier.com), licensed under a Creative Commons Attribution 3.0 Unported License (https://creativecommons.org/licenses/by/3.0).

FIG. 3 depicts the concentrations of growth factors as found in the supernatants of cell cultures in flasks determined by multiplex analysis. Values were normalized to the density 10.000 cells of the corresponding culture.

FIG. 4 shows the formation of new dermal tissue in wounds of pigs treated with freeze dried primary human keratinocytes grown without (control) or with the rock inhibitor Y27632 compared to the placebo formulation without any cells (negative control) or the cell based wound dressing epifast (positive control, keratinocyte-based dressing, not freeze dried, approved and used to treat burns in Mexico). Shown are the areas of newly formed dermis quantified by morphometry of hematoxilin-/eosin-stained wound sections.

### DETAILED DESCRIPTION OF THE INVENTION

The present invention is now described in more detail by preferred embodiments and examples, which are however presented for illustrative purposes only and shall not be understood as limiting the scope of the present invention in any way.

The present invention refers to a cell having the capacity of producing at least one SASP factor, wherein this cell is obtained or obtainable by treating (modifying) the cell with an amount of an exogenous secretion inhibitor that is sufficient to reduce, preferably inhibit, the secretion of the at least one SASP-factor. By treating the cell with a sufficient amount of exogenous secretion inhibitor, the secretion of SASP factor(s) from the inside of the cell to the outside of the cell is reduced, preferably inhibited, thereby resulting in a cell that, due to the inhibited secretion of the SASP factor(s) out of the cell, is or becomes highly enriched or loaded and preferably super-loaded in these SASP factor(s). Once such a treated/modified cell is applied or further transferred into a composition for use, the thus achieved high amount of loaded SASP/growth factors upon application will then release/deliver and thereby beneficially contribute to therapeutic effects, such as the therapeutic effects described in further detail below.

It has been found in the present invention that cells that have the capability of producing SASP factors and that have been treated with exogenous secretion inhibitors are not able anymore, entirely or at least at a substantially reduced degree, to secrete the SASP factors that have been or are produced (e.g., produced upon intentional stimulation of the cell). It is assumed in the present invention that this, in turn, results in an endogenous enrichment of said SASP factor(s). It is further assumed in the present invention that these cells that are endogenously highly enriched in SASP factor(s) (“super-loaded” cells) are able to release these SASP factor(s) e.g., if the cells are applied to a treatment site such as a wound. It has been shown in the present invention that said cells which secrete SASP factors at an at least substantially reduced degree, if applied to a wound, result in an improved wound healing.

It is thus currently hypothesized by the inventors that the treating (modification) of the cells that have the capability of producing at least one SASP factor with an amount of an exogenous secretion inhibitor that is sufficient to reduce, preferably inhibit, the secretion of the at least one SASP factor, results in an endogenous enrichment of said SASP factor(s). It is further hypothesized that, if the resulting endogenously enriched cell is applied to a wound, e.g. in combination with a suitable carrier and/or one or more further active agent(s), said cell releases the endogenously enriched SASP factor(s) to the wound, thereby resulting in an improved wound healing.

A release of said endogenously enriched SASP factor(s) can take place via holes of the cell membrane, or by cell disintegration, respectively in a passive manner. If the cells accordingly to the present invention that are endogenously highly enriched in SASP factor(s) are then, for example after freeze drying, applied to the therapeutic site of interest, can optionally reconstitute in the associated body fluids such as in the wound and thereby release the SASP factor(s), which subsequently exhibits the therapeutic effect, e.g. beneficially contributing to wound healing.

So far, and contrary to the present invention, in prior art viable cells are used that actively secrete different SASP factors/growth factors. The cells in the prior art are not treated with secretion inhibitors to prevent protein secretion or secretion of SASP factors, respectively. As a result, these prior art cells are not enriched, let alone highly enriched or super-loaded, with SASP factor(s)/growth factor(s) compared to cells in the prior art that are not treated with secretion inhibitors to prevent protein secretion or secretion of SASP factors, respectively.

The term “SASP” denotes a senescence-associated secretory phenotype (“SASP”) which is characterized in that cells that have this phenotype e.g., upon suitable stimulation, produce and secrete proteins or polypeptides that constitute or represent the SASP. The SASP factors may include several families of soluble and insoluble factors, which, upon secretion, are able to influence and affect surrounding cells. The SASP, also termed senescence-messaging secretome, may include the expression/secretion of the following biologically active factors [9]:
i. Interleukins, such as IL-1α, IL-1β, IL-6, IL-7, IL-13, IL-15;
ii. Chemokines, such as IL-8, MCP2, MCP4, GROα, GROβ, GROγ;
iii. Growth factors, such as EGF, HGF, VEGF;
iv. Receptors and ligands, such as ICAM1, ICAM3, TRAIL-R3, Fas, uPAR, sTNFRI, sTNFRIII;
v. Proteases and regulators, such as MMP1, MMP3, MMP10, MMP12, TIMP1, TIMP2, PAI1, PAI2,
vi. Extracellular insoluble molecules, such as Collagens, Fibronectins, Laminins.

In the present invention, in one embodiment, the cells have the capability of producing at least one SASP factor selected from the group consisting of IL-1α, IL-1β, IL-6, IL-7, IL-8, IL-10, IL-13, IL-15, IL-18, MCP1, MCP2, MCP4, MIF, MIP-1a, MIP-3a, HCC-4, Eotaxin-3, TECK, ENA-78, I-309, I-TAC, GROα, GROβ, GROγ, VEGF, EGF, HGF, HBEGF, FGF, such as FGF acidic, bFGF, KGF, NGF, Amphiregulin, Angiogenin, APOJ, CAV1, OSTEO, Epiregulin, Heregulin, SCF, SDF-1 alpha, PIGF, IGF-1, IGFBP-1, -2, -3, -4, -5, -6, -7, GM-CSF, PDGF-BB, TGF-α, TGF-β1, TGF-β2, TGF-β3, TRAIL-β3, Fas, OPG, SGP130, EGF-R uPAR, sTNFRI, sTNFRIII, MMP1, MMP2, MMP3, MMP7, MMP9, MMP10, MMP12, MMP13, MMP14, TIMP1, TIMP2, PAI1, PAI2, Park7 DJ-1, uPA/Urokinase, SLPI, SPP1, Syndecan 1, -4, Tenascin C, Endothelin, Collagens, Fibronectins, Laminins, ICAM1, and ICAM3.

In another embodiment, the cell is intracellularly (endogenously) enriched in at least one or more of the following SASP factors: IL-1 alpha, IL-6, IL-8, TGF-α, GROα, VEGF, EGF, FGF acidic, MMP-7, TIMP1.

In still another embodiment, the cell is intracellularly (endogenously) enriched in at least one or more of the following SASP factors belonging to the group of cytokines or chemokines: IL-1α, IL-1β, IL-6, IL-7, IL-8, IL-10, IL-13, IL-15, IL-18, MCP1, MCP2, MCP4, MIF, MIP-1a, MIP-3a, HCC-4, Eotaxin-3, TECK, ENA-78, I-309, I-TAC, GROα, GROβ, GROγ.

In still another embodiment, the cell is intracellularly (endogenously) enriched in at least one or more of the following SASP factors belonging to the group of growth factors: VEGF, EGF, HGF, HBEGF, FGF, such as FGF acidic, bFGF, KGF, NGF, Amphiregulin, Angiogenin, APOJ, CAV1, OSTEO, Epiregulin, Heregulin, SCF, IGF-1, IGFBP-1, -2, -3, -4, -5, -6, -7, GM-CSF, PDGF-BB, TGF-α, TGF-β1, TGF-β2, TGF-β3.

In still another embodiment, the cell is intracellularly (endogenously) enriched in at least one or more of the following SASP factors belonging to the group of proteases and their inhibitors: MMP1, MMP2, MMP3, MMP7, MMP9, MMP10, MMP12, MMP13, MMP14, TIMP1, TIMP2, PAI1, PAI2, Park7 DJ-1, uPA/Urokinase, SLPI.

In still another embodiment, the cell is intracellularly (endogenously) enriched in at least one or more of the following SASP factors belonging to the group of structural proteins found in or attached to cellular membranes: Syndecan 1, Syndecan-4, Tenascin C, Endothelin, Collagens, Fibronectins, Laminins, ICAM1, and ICAM3.

The cells' capability of producing at least one SASP factor can be tested by any suitable test method that is known to a skilled person. For example, after having stimulated cells suitably (that is, stimulated in a way that results in production of SASP factor(s)), the presence of said SASP factor(s) can e.g. be determined in the supernatants from the cell culture flasks or/and proteins extracts from the stimulated cells. For this purpose, the supernatant is collected or protein extracts are generated from the cells and protein concentration can be determined and if desired quantified e.g. by using the Bio-Plex® Multiplex Immunoassays or ELISA kits from BIO-RAD. Intracellular levels of SASP factor(s) can be determined e.g. by lysis of a cell layer using e.g. the Bio-Plex™ Cell Lysis Kit from BIO-RAD. Once a cell may be selected by its determination or confirmation that the cell is capable of producing at least one SASP factor, such selected cell can be used for being treated/modified according to the present invention.

Cells that have the capacity of producing at least one SASP factor are in general cells that are able, e.g. upon stimulation, to produce one or more SASP factor(s). In a preferred embodiment, cells that have this capacity are selected from the group consisting of keratinocytes, fibroblasts, epithelial cells, melanocytes, endothelial cells, corneal epithelial cells, pericytes, monocytes, lymphocytes, thrombocytes, mast cells, adipocytes, muscle cells, neurons, osteocytes, osteoblasts, chondrocytes, mesenchymal stem cells, and adult or embryonic stem cells, preferably the cells are selected from the group consisting of epithelial cells, corneal epithelial cells, keratinocytes, fibroblasts, melanocytes, endothelial cells, pericytes, monocytes, lymphocytes, thrombocytes, mast cells, adipocytes, mesenchymal stem cells, and/or adult or embryonic stem cells, more preferably, the cell is a skin-associated cell, particularly the cell is selected from the group consisting of keratinocytes, fibroblasts, adipocytes and endothelial cells. In a particularly preferred embodiment, the cell that is capable of producing at least one SASP factor is a keratinocyte.

An example of cells that are capable of producing SASP factor(s) are cells having a senescent phenotype. “(Cellular) senescence” is a mechanism that occurs in vivo and in vitro locking the cell into a cell cycle arrest, and in this conventional context was known to inhibit malignant transformation and to contribute to aging. A wide range of different stress factors are reported to trigger cellular senescence [10, 11]. These include telomeric dysfunction upon repeated cell division (replicative senescence), oxidative stress, mitochondrial deterioration, severe or irreparable DNA damage and chromatin disruption (genotoxic stress) and the expression of certain oncogenes [12-14]. Besides its well-established role as being a potent tumor suppressive mechanism and a contributor to aging, there is evidence for senescent cells developing altered secretory activities thus promoting tumorigenesis [15, 16]. For example, Coppé and colleagues described, that fibroblasts having a senescent-associated secretory phenotype thus turning senescent fibroblasts into proinflammatory cells that have the ability to promote tumor progression [17]). Recently, a fourth role of senescence has been emerged, when Krizhanovsky et al. discovered that tissue damage-induced hyperproliferation of hepatic stellate cells (HSCs) induces cell senescence leading to a reduction in the secretion of extracellular matrix (ECM) proteins and enhanced secretion of ECM degrading proteins, thereby limiting fibrosis upon tissue damage within the liver [18, 19]. Preferably, “senescence” shall not be simply reduced to cell cycle arrest. Rather, senescence can be caused by growth stimulation, when the cell cycle is arrested ([20]). Thus, as one further hallmark, senescent cells may lose the potential to resume proliferation.

However, a senescent cell is a potentially persisting cell that is metabolically active and has undergone extensive changes in the protein expression and secretion pattern, thus finally developing its individual SASP resembling a kind of fingerprint [17]. This fact may explain the four obviously opposing functions of senescent cells mentioned above highlighting the importance of the cellular context, i.e. cell type and senescence-inducing stimulus [17, 21].

In addition to the above mentioned cell cycle arrest and the specific SASP, a senescent cell may be characterized by the following hallmarks [9]:
a. Enlarged, flattened morphology
b. p16^{INK4a} expression
c. elevated lysosomal activity (senescence-associated b-galactosidase; SA b-gal)
d. DNA-damage response
e. chromatin remodelling
f. autophagy.

For enhancing the expression of SASP factor(s) and enriching as much SASP factors in the cell as possible, the cell is preferably stimulated to start producing SASP factors. This can for example be achieved by a mitogenic stimulus or a specific exogenic stimulus that stimulates a signal transduction pathway directly or indirectly stimulating the expression of the SASP factor. Suitable stimuli are known to a person skilled in the art.

Cells that are senescent also produce SASP factors. Hence, upon inducing senescence, cells also produce SASP factors. If for example cells are culture in vitro, senescence can be induced either by inappropriate growth conditions leading to cell culture stress, for example overgrowth of the cells, high cell density, cell contact inhibition, presence of H₂0₂, presence of ROS, administration of mitomycin C or any other chemically-based mitotic inhibitor, irradiation with γ-Rays, irradiation with X-Rays, or irradiation with UV light, particularly UVB as for example described by [22], and treatment with cold plasma [23].

In another embodiment of the present invention, senescence may be induced by oxidative stress/reactive oxygen species, radiation or e-beam treatment.

Furthermore, senescence can be induced by sublethal doses of surfactants, for example NP-40, preferably in a concentration of 1-10 μM, Triton X-100, preferably in a concentration of 2-20 μM, SDS, Tween 20, Tween 80, preferably in a concentration of 5-20 μM, cardiolipin, and soap. For inducing senescence, cells are cultured with surfactants in sublethal concentrations, for example for one to six weeks.

Alternatively, senescence may be induced by a low-dose ionizing radiation as for example described by [24-27].

Alternatively, relatively low levels of Mitomycin C have been found to be senescence-inducing, for example in a concentration of 0.02-1 μM. Preferably, the cell culture medium including Mitomycin C in the above-mentioned concentrations is changed for several times, for example for 2 to 7 time, for example every day to every third day. Then, also slightly lower doses of Mitomycin C may induce senescence.

Alternatively, t-BHP or Ethanol and small molecule inducers of senescence in suitable concentrations, as for example described elsewhere [5, 28-30] may be used for inducing senescence.

The respectively used method is controlled, for example by using appropriate concentration and treatment duration of the respective agent or appropriate intensity and duration of the respective physical treatment, to the extent that the senescent cell cycle arrest is attained, which can be verified by any one of the above described characteristics of senescence or SASP.

In a preferred embodiment, senescence may be induced, e.g. by any one of the above methods or treatments in the presence of a sustained growth stimulus, i.e. the growth stimulus is applied concurrently to cell cycle arrest. In other words: senescence according to this invention may be induced by coupling cell cycle arrest, preferably by contact inhibition with a growth stimulus. Such growth stimulus according to the invention, may be selected from the means consisting of: media components like serum or serum components in particular from xenogeneic or human source, platelets, platelet lysates or components of the later as well as single or combination of growth factors like for example EGF, KGF, FGF, insulin, hydrocortisone or apotransferrin in concentrations known by a person skilled in the art. A further growth stimulus may be TGF-ß.

Moreover, cell cycle arrest/proliferation may be analyzed by methods known in the art, for example DNA stains involving 3H-thymidine or BrdU or staining of cell cycle regulators.

According to the present invention the thus described and/or selected cell is treated with an amount of an exogenous secretion inhibitor that is sufficient to significantly reduce, preferably inhibit, the secretion of the at least one SASP factor. An exogenous secretion inhibitor is a compound that is not present endogenously in the cell, but that is added from outside the cell.

A skilled person is able to determine whether the amount of exogenous secretion inhibitor is suitable (sufficient) to at least significantly reduce, preferably inhibit, the secretion of the at least one SASP factor. For this purpose, the intracellular and/or extracellular amount of a specific SASP factor of cells that have been treated with the secretion inhibitor and of cells that have not been treated with this secretion inhibitor, can be determined and compared. A suitable method for determining the amount of SASP factor, extracellularly (e.g., in the supernatant) as well as intracellularly (e.g., from cell lysates/cell extracts) is disclosed elsewhere herein.

As a result of this treatment of the cell with the exogenous secretion inhibitor, the treated cell
(i) exhibits a cellular structure or cellular function that is different to the cellular structure or cellular function without the respective treatment;
(ii) exhibits reduced ability to secrete the at least one SASP-factor, preferably is not able to secrete the at least one SASP factor anymore; and, as a result from the treatment with the exogenous secretion inhibitor; and/or
(iii) is intracellularly enriched in the at least one SASP-factor.

In a preferred embodiment, as a result of this treatment of the cell with the exogenous secretion inhibitor, the treated cell
(i) exhibits a cellular structure or cellular function that is different to the cellular structure or cellular function without the respective treatment;
and
(ii) exhibits reduced ability to secrete the at least one SASP-factor, preferably is not able to secrete the at least one SASP factor anymore, and/or
(iii) is intracellularly enriched in the at least one SASP-factor.

Hence, it is also possible that the treated cell, as a result of the treatment with the exogenous secretion inhibitor, exhibits capacity (i), and additionally (ii) or (iii). In a preferred embodiment, the treated cell exhibits at least capacity (i) and (iii), preferably (i), (ii), and (iii).

In order to determine whether a cell has suitably been treated with an exogenous secretion inhibitor in accordance with the present invention, feature (capacity) (i) as disclosed above, in combination with feature (ii) and/or (iii), respectively as disclosed above, or feature (i) in combination with feature (ii), preferably all features (i) to (iii), should apply. In this context, feature (iii) may be judged in relation to the cell status before the treatment.

Upon treating the cell with the exogenous secretion inhibitor in an amount that is sufficient in order to significantly reduce, preferably to inhibit, secretion of SASP factor(s), typically cellular structures and/or cellular functions are affected, to the effect that secretion of SASP factor(s) is significantly reduced, preferably inhibited. In other words, due to the treatment with the exogenous secretion inhibitor, particular structures and/or functions of the cell are not able to exert their usual (wildtype) function anymore.

The cellular structure or cellular function that is different to the respective cellular structure or function without the respective treatment is one or more selected from the group consisting of:
(i) function of protein insertion into the endoplasmic reticulum (ER);
(ii) function of protein maturation;
(iii) function of protein trafficking and/or vesicle formation;
(iv) function of microtubules; and
(v) function of kinesin; and/or
(vi) function of autophagy.

Each of these groups can be affected by a corresponding compound, such as selected from the following described Compound Group I, Compound Group II, Compound Group III, Compound Group IV, Compound Group V, and Compound Group VI, to the effect that finally the secretion of SASP factors is significantly reduced, preferably inhibited.

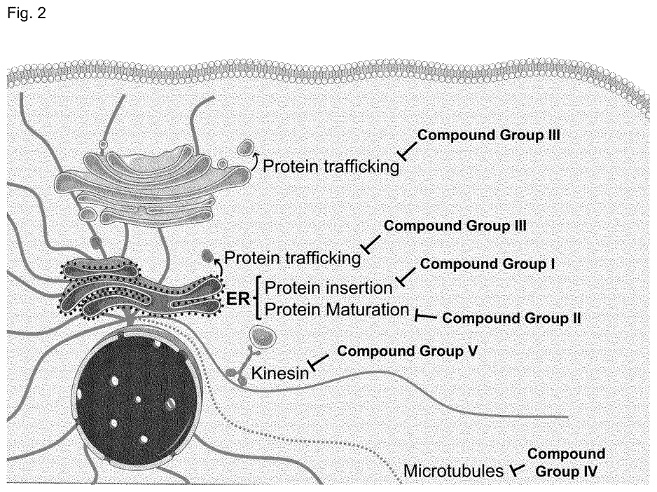

FIG. 2 represents an overview of the cellular structures and functions, and shows where the respective compounds exhibit their effect. “Compound Group VI” is not shown in FIG. 2.

The respective exogenous secretion inhibitors (“Compound Group I-VI”) as well as their mode of action are as follows:

### Protein Insertion Endoplasmic Reticulum (ER) (Compound Group I)

The compounds (secretion inhibitors) of group I are secretion inhibitors that affect the protein insertion (import) in the ER.

After protein translation, the newly synthesized proteins (e.g., the SASP factor(s)) enter the ER lumens or are inserted into the ER membrane. Several molecules target Sec61 protein, a translocation channel responsible for ER protein translocation [8, 31]. Examples for secretion inhibitors that affect protein insertion into the ER are Mycolactone; Cotransisns, e.g. HUN-729; Apratoxins, e.g. Apratoxin A, Decatransin, Coibamide A, Ipomeassin-F, and Spiperone.

### Protein Maturation in ER (Compound Group II)

The ER acts folding the newly synthesized proteins. A high production of proteins can lead the ER to enter a stress situation, where the number of synthesized proteins surpass the ER capacity to fold them. This stress situation triggers the unfolded protein response (UPR), which will lead to a process that will ultimately restore the ER's folding balance. ER's folding machinery is luminal Ca²⁺ dependent. By the inhibition of the sarco/endoplasmic reticulum Ca²⁺-ATPase (SERCA), a state of Ca²⁺ depletion can be achieved, stopping the ER's folding process and leading to a protein accumulation [32]. Examples for secretion inhibitors that affect protein maturation in the ER are Sesquiterpene lactones, e.g. thapsigargin; Basiliolides, e.g. Basiliolide; Inhibitors of protein glycosylation, e.g. Tunicamycin; and Compounds blocking the eukaryotic signal peptidase, e.g. Cavinafungin.

Protein Trafficking (the Transport and Movement of Proteins to their Correct Subcellular Compartments or to the Extracellular Space; Also Referred to as “Secretory Pathway”) (Compound Group III)

Under the compound group III fall the so-called rock inhibitors, which are for example described by Lee et al. or Chapman et al. [33, 34]. For instance, ADP-ribosylation factor (ARF) proteins are important to the protein secretion and thus an effective target of group III compounds. These proteins regulate cellular traffic through GDP/GTP cycling. ARF activation is facilitated by the guanine nucleotide exchange factors (GEF), which forms an ARF-GEF complex. Some molecules are bound to the ARF-GDP-Sec7 domain of the ARF-GEF complex, inhibiting the further regulation of the vesicle formation. This ultimately led to the release of the coat protein into the cytosol. The effect of this inhibitor molecule is to stop the secretion of the expressed protein [8, 35]. Examples of secretion inhibitors that affect the secretory pathway are Brefeldin A; and vesicle formation inhibitors, e.g. Calpastatin, Calpeptin, Calpain inhibitor, Manumycin A, GW4689, and Y27632.

### Function of Microtubules (Microtubules Inhibitors) (Compound Group IV)

Microtubules are structural elements of Eukaryotic cells and part of the cytoskeleton. This network plays a key role migration, mitosis, etc. Microtubules also control the intracellular traffic of proteins, which is also key to the cell secretory process. Composed by polymers of α- and β-tubulin, microtubules are in a constant process of growing and shortening, known as dynamic instability. They can be affected by inhibitors in two ways: through the stabilization of the structure or through the destabilization. Some molecules have the capacity to bind to one of the tubulins and prevent the disassembly, hence stabilizing the network. Other molecules, which also binds to tubulin monomers, prevent the polymerization, which will lead to the destabilization of the structure [36, 37]. Examples of secretion inhibitors that affect the function of the microtubules are
microtubule-destabilizing agents with vinca-domain,
e.g. Vinblastin, Vincristine, Vindesine, Vinorelbine, Eribulin, E7974, Tubulsin A, Cryptophycin 52, Halichondrins such as E7389, Dolastatins such as TZT-1027, Hemiasterlins such as HTI-286;
microtubule-destabilizing agents with colchicine domain,
e.g. Colchicine, BPROLO75, Indublin, Combretastatins e.g. AVE8062A, CA-1-P, CA-4-P, N-acetylcolchicinol-O-phosphate, ZD6126; 2-Methoxyestradiol; Methoxybenzene-sulphonamide such as ABT-751, E7010; 2-methoxy-5-(2,3,4-trimethoxyphenyl) 2,4,6-cycloheptatrien-1-one;
microtubule-stabilizing agents with taxane domain,
e.g. taxanes such as Paclitaxel (Taxol), TL00139 and analogues thereof, docetaxel (Taxotere), Epothilones such as BMS-247550, Epothilones B and D, Discodermolide;
microtubule-stabilizing agents with laulimalide domain,
e.g. Epothilones, such as Desoxyepothilone B; and
microtubule-assembly inhibitors,
e.g. Astemizole, Parbendazole;

### Kinesin Inhibitors (Compound Group V)

Kinesins are motor proteins that bind to microtubules and moves towards the plus end through the hydrolysis of ATP. Some molecules selectively inhibit kinesin spindle protein (KSP) from Kinesin, which prevents the binding of the protein to the microtubule structure, blocking cell mitosis [38]. Examples of secretion inhibitors that affect kinesin function are Ispinesib (SB715992); SB743921 (hydrochloride); GSK923295 (an allosteric inhibitor of centromere-associated protein-E (CENP-E) kinesin motor ATPase activity); GW406108X (a Kif15 inhibitor); Dimethylenastron; ARQ 621 (inhibitor of Eg5, a microtubule-based ATPase motor protein involved in cell division).

Compounds that Affect Autophagy (Compound Group VI)

Autophagy is a natural degradation of the cell that removes unnecessary or dysfunctional components. If autophagy is affected, this can result in an inhibition of secretion. Examples of secretion inhibitors that affect the function of autophagy are autophagy inducers, such as the autophagy inducers listed in Table 1, and autophagy inhibitors, such as the autophagy inhibitors also listed in Table 1.

In this Table 1, there is also indicated the respective concentration (amount) of the respective secretion inhibitor that is necessary in order to arrive at a significant reduction, preferably inhibition, of the secretion. It is possible that a specific compound (secretion inhibitor) exhibits multiple functions, so that this compound can be listed in two or more groups. There are also compounds that are not categorized in a specific group I to VI. Table 1 also indicates the concentration range that results in a significant reduction, preferably inhibition, of SASP factor secretion.

TABLE 1
Inhibitors or modulators of protein secretion. IC₅₀ refers to the half of
the maximal concentration necessary to inhibit the microtubules assembly.

| | | | |
|---|---|---|---|
| Class | Substance | Concentration | Ref. |

ER import inhibitors: Group I

| | Mycolactone | 0.02/0.2 μM-2.0/20 μM | | [39, 40] |
|---|---|---|---|---|
| Cotransins | HUN-7293 | 0.1/1 μM-10.0/100 μM | | [41] |
| Apratoxins | Apratoxin A | 0.01/1 μM-1/10 μM | | [42] |
| | Decatransin | 0.1/1 μM-10.0/100 μM | | [43] |
| | Coibamide A | 0.1/1 μM-10.0/100 μM | | [44] |
| | Ipomeassin-F | 0.01/1 μM-1/10 μM | | [45] |
| | Spiperone | 10-20 | μM | [46] |

Protein maturation inhibitors: Group II

| | | | |
|---|---|---|---|
| Sesquiterpene lactones | Thapsigargin | 10/50 nM-100/200 nM | [47, 48] |
| Basiliolides | Basiliolide | 0.1/0.5 μM-5/20 μM | [49] |
| Inhibitors of protein | Tunicamycin | 1/5 μg/ml-25/50 μg/ml | [50, 51] |
| glycosilation | | | |
| Compounds blocking the | Cavinafungin | 1/10 nM-100/900 nM | [52] |
| eukaryotic signal | | | |
| peptidase | | | |

Trafficking inhibitors: Group III

| | | |
|---|---|---|
| Brefeldin A | 0.1/1 μM-10.0/100 μM | [53] |

Vesicle formation inhibitors: Group III

| Calpastatin | 5/15 μM-20/50 μM | | |
|---|---|---|---|
| Calpeptin | 80 μM (1 mM | | [54] |
| | extracellular Ca² - | | |
| | calcium dependent) | | |
| Calpain inhibitor II | 10 | μM | [55] |
| Manumycin A | 1 | μM | [54] |
| GW4689 | 0.5-20 | μM | [54] |
| Y27632 | 5 | μM | [54] |

Lipid metabolism inhibitors

| | Pantethine | 0.1/0.5-1.0 mM/5 mM | | |
|---|---|---|---|---|
| | Imipramine | 1 | μM | |
| Tubulin inhibitors: | | | | [56] |
| Group IV | | | | |
| Microtubule-destabilizing | Vinblastine, Vincristine, | 0.8 | nM (IC₅₀) | [57-59] |
| agents-vinca domain | Vindesine, Vinorelbine, | | | |
| | Vinflunine; | | | |
| | Eribulin; | | | |
| | E7974; | | | |
| | Tubulsin A; | | | |
| | Cryptophycin 52; | | | |
| | Halichondrins (such as | | | |
| | E7389); | | | |
| | Dolastatins (such as | | | |
| | TZT-1027); | | | |
| | Hemiasterlins (such as | | | |
| | HTI-286); | | | |
| Microtubule-destabilizing | Colchicine | 2-5 | μM | [60] |
| agents-colchicine domain | Combrestatins: | | | |
| | BPR0L075; | | | |
| | ABT-751; | | | |
| | Indibulin; | | | |
| | Combretastatins | | | |
| | (AVE8062A, CA-1-P,CA- | | | |
| | 4-P, N-acetylcolchicinol- | | | |
| | O-phosphate, ZD6126); | | | |
| | 2-Methoxyestradiol; | | | |
| | Methoxybenzene- | | | |
| | sulphonamide (such as | | | |
| | ABT-751, E7010); | | | |
| | 2-methoxy-5-(2,3,4- | | | |
| | trimethoxyphenyl) 2,4,6- | | | |
| | cyc!oheptatrien-l-one; | | | |
| Microtubule-stabilizing | Taxanes: | 8 | nM (IC₅₀) | [59] |
| agents-taxane domain | Paclitaxel (Taxol), | | | |
| | TL00139 and other | | | |
| | analogues | | | |
| | Docetaxel (Taxotere) | | | |
| | Epothilones (such as | | | |
| | BMS-247550, | | | |
| | epothilones B and D) | | | |
| | Discodermolide | | | |
| Microtubule-stabilizing | Epothilones: | 13 | nM (IC₅₀) | [61] |
| agents-epothilone domain | Desoxyepothilone B | | | |
| Microtubule-stabilizing | Laulimalide | 50 to 100 | nM | [62] |
| agents-laulimalide domain | Peloruside A | | | |
| Microtubule-assembly | Astemizole | 20-40 | μM | [46] |
| inhibitor | Parbendazole | 10 | μM | [46, 63] |
| Kinesin inhibitors: | | | | [64, 65] |
| Group V | | | | |
| | Ispinesib (SB715992) | 10-30 | μM (IC₅₀) | [64] |
| | SB743921 | | | |
| | GSK923295 | | | |
| | GW406108X | | | |
| | Dimethylenastron | | | |
| | ARQ 621 | | | |
| Autophagy inducers: | | | | [66] |
| Group VI | | | | |
| Inhibitor of mTORC1 | Rapamycin | 63 | pM (IC₅₀) | [67, 68] |
| | Sirolimus | 75 | pM (IC₅₀) | [68] |
| | Temsirolimus | | | |
| | Everolimus | | | |
| | Deforolimus | | | |
| | Perhexiline | | | |
| | Niclosamide | | | |
| | Amiodarone | | | |
| | Rottlerin | | | |
| | DL001 | | | |
| Inhibitor of mTORC1/ | Torin1 | 250 | nM (IC₅₀) | [69] |
| mTORC2 | AZD8055 | | | |
| Dual inhibitor of PI3K- | PI-103 | 8-150 | nM (IC₅₀) | [70] |
| mTOR | NVP-BEZ235 | | | |
| Inhibitor of tyrosine kinase | Imatinib | | | |
| | Dasatinib | | | |
| Inhibitor of proteasome | Bortezomib | 4-800 | nM (IC₅₀) | [71] |
| | NPI-0052 | | | |
| Inhibitor of histone | LAQ824 | 10-100 | nM (IC₅₀) | [72] |
| deacetylase | Vorinostat | | | |
| | Panobinostat | | | |
| | OSU-HDAC42 | | | |
| BH3 domain mimetics | ABT-737 | 5-10 | μM (IC₅₀) | [73] |
| | HA14-1 | | | |
| Inhibitor of IP3 | L-690,330 | 100 | μM | [74] |
| IP3 receptor antagonist | Xestospongin B | 0.08-10 | μM | [74, 75] |
| Activator of AMPK | Metformin | 5 | μM | [76] |
| Antiestrogen | Tamoxifen | | | |
| Alkylating agent | Temozolomide | 50 | μM* | [77] |
| Antioxidant | Resveratrol | 12.5-50 | μM | [78] |
| Antidepressant | Fluoxetine | 30-100 | μM | [79] |
| | Maprotiline | | | |
| Inhibitor of glycolysis | 2-Deoxyglucose | 50 | mM | [80] |

| | | | |
|---|---|---|---|
| Inhibitor of | Lonafarnib | 5 μM (5 μM + 4 μM | [81, 82] |
| farnesyltransferase | | Lonafarnib + | |
| | | Sorafenib)† | |
| Pan-RAF inhibitor | Sorafenib | 5-10 μM (4 μM + 5 | [82] |
| | | μM Sorafenib + | |
| | | Lonafarnib)† | |

Autophagy inhibitors: Group VI

| Inhibitors of class III PI3K | LY294002 | 2/4 μM-20/50 μM | | |
|---|---|---|---|---|
| | 3-Methyladenine | 5 | mM | |
| Inhibitor of the | Chloroquine | 6-12 μM/100 μM | | [83, 84] |
| acidification of lysosome | Hydroxychloroquine | | | |
| | Bafilomycin A1 | | | |
| Inhibitor of lysosomal | Leupeptin | 80-96 | nM | [85] |
| proteolysis | Pepstatin A | | | |
| Inhibitor of microtubule | Nocodazole | 0.8 | nM (IC₅₀); | [57-59] |
| formation | Colchicine | | | |
| | Vinblastine | | | |
| Inhibitor protein transport | Monensin | 10 | μM | [84] |

| | |
|---|---|
| Inhibitor of ubiquitin- | Spautin-1 |
| specific peptidases | |

Senescent factor with secretion inhibition

| immunosuppressive | Cyclclosporine A | 10-40 | μM | [46, 86] |
|---|---|---|---|---|
| calcineurin inhibitor) | | | | |
| Calcium channel inhibitor | Fendiline | 1-100 | μM | [46, 87] |

Other mechanisms

| Antidepressant | Paroxetine | 10-20 | μM | [46] |
|---|---|---|---|---|
| | Sertraline | 10 | μM | [46] |
| effect on signal | Parthenolide | 10-20 | μM | [46] |
| transduction pathway | | | | |
| inhibitor of mitochondrial | Perhexiline | 10 | μM | [46] |

carnitine
palmitoyltransferase-1

Combination of actin depolymerization inductors
and Microtubule targeting-agents

| | | | |
|---|---|---|---|
| Actin depolymerizer | Methyl-β-cyclodextrin | 1 mM + 2-5 nM | [57] |
| | | Vinblastine | |
| Synergistic effect | | | [62, 88] |
| Microtuble inhibition | Laulimalide + 2- | 1.75 nm + 250 nM | |
| laulimalide domain | methoxyestradiol; | (over IC₅₀) | |
| | Laulimalide + Paclitaxel; | 1.3 nM + 1.1 nM | |
| | C16-C17-des-epoxy | (IC₅₀) | |
| | laulimalide + 2- | 150 nM + 150 nM | |
| | methoxyestradiol; | (over IC₅₀) | |
| | C16-C17-des-epoxy | 100 nM + 1 nM | |
| | laulimalide + Paclitaxel; | (over IC₅₀) | |
| | C20-methoxy laulimalide + | 290 nM + 150 nM | |
| | 2-methoxyestradiol; | (IC₅₀) | |
| | C20-methoxy laulimalide + | 250 nm + 1 nm | |
| | Paclitaxel; | (over IC₅₀) | |

*human glioma cell lines (induce senescence; alkylating agent for DNA in glioma therapy).
†Synergistically inhibition found when combined Lonafarnib and Sorafenib).

The respective group indicated (group I-VI) is also reflected in FIG. 2.

It is known to a person skilled in the art that a certain exogenous compound can have a certain (known) effect on the cellular structure and/or function. Therefore, it is possible to conclude upon analysing a cell with regard to its cellular structure and/or cellular function that it has been treated (modified) with an exogenous secretion inhibitor of a specific group as disclosed herein, or a further secretion inhibitor that is a member of a specific class e.g. as disclosed in table 1 herein.

In an embodiment, the cell that is capable of producing at least one SASP factor is a senescent cell type. Preferably, this senescent cell type derives from cells being selected from the group consisting of keratinocytes, fibroblasts, epithelial cells, melanocytes, endothelial cells, corneal epithelial cells, pericytes, monocytes, lymphocytes, thrombocytes, mast cells, adipocytes, muscle cells, neurons, osteocytes, osteoblasts, chondrocytes, mesenchymal stem cells, and adult or embryonic stem cells, preferably the cells are selected from the group consisting of epithelial cells, corneal epithelial cells, keratinocytes, fibroblasts, melanocytes, endothelial cells, pericytes, monocytes, lymphocytes, thrombocytes, mast cells, adipocytes, mesenchymal stem cells, and/or adult or embryonic stem cells, more preferably, the cell is a skin-associated cell, particularly the cell is selected from the group consisting of keratinocytes, fibroblasts, adipocytes and endothelial cells.

In a further embodiment, it is also possible that the cell is intracellularly loaded or enriched in at least one SASP-factor selected from the group consisting of IL8, GRO alpha, VEGF, Endothelin, MMP7, MMP9. MMP10, MMP12, MMP13, TIMP1, TIMP2, and TGF beta 1. Preferably the intracellular concentration, calculated as pg/mg protein extract, of said one or more SASP factor, if present, is defined as follows:

| | intracellular | | | |
|---|---|---|---|---|
| | (pg/mg protein | | | |
| | extract) | optional | optional | optional |
| IL8 | more than 20 | more than 20, | 50-1.000 | 50-250 |
| | | up to 1000 | | |
| GRO alpha | more than 20 | more than 20, | 50-1.000 | 50-250 |
| | | up to 1000 | | |
| VEGF | more than 20 | more than 20, | 50-1.000 | 50-250 |
| | | up to 1000 | | |
| Endothelin | more than 20 | more than 20, | 50-1.000, | more than |
| | | up to 1000 | preferably | 100, up to- |
| | | | more than | 250 |
| | | | 100 to 1.000 | |
| MMP7 | more than 200 | more than, up | 2.000-20.000 | more than |
| | | 200 to 20.000 | | 10.000, up |
| | | | | to 20.000 |
| MMP9 | more than 200 | more than | 2.000-20.000 | more than |
| | | 200, up to | | 10.000, up |
| | | 20.000 | | to 20.000 |
| MMP10 | more than | more than | 10.000-40.000 | more than |
| | 2.000 | 2.000, up to | | 20.000, up |
| | | 40.000 | | to 40.000 |
| MMP12 | more than 10 | more than 10, | 40-200 | more than |
| | | up to 200 | | 100, up to |
| | | | | 200 |
| MMP13 | more than 100 | more than | 400-1.000 | |
| | | 100, up to | | |
| | | 1.000 | | |
| TIMP1 | more than 200 | more than | 2.000-20.000 | more than |
| | | 200, up to | | 10.000, up |
| | | 20.000 | | to 20.000 |
| TIMP2 | more than 200 | more than | 2.000-20.000 | more than |
| | | 200, up to | | 10.000, up |
| | | 20.000 | | to 20.000 |
| TGF beta 1 | more than 10 | more than 10, | 40-2.000 | more than |
| | | up to 2.000 | | 1.000, up to |
| | | | | 2.000 |

In the context of the present invention, the expression “more than”, followed by a specific number, defines that this specific number directly after the expression “more than” is excluded.

In a further embodiment, the cell of the present invention may be frozen, spray-dried or freeze-dried or is present in or transferred into a frozen, dried, spray-dried or freeze-dried composition, or a composition derived from said cell. Suitable methods for freezing, spray-drying or freeze-drying are known to a skilled person.

The present invention further refers to a composition comprising or being derived from the cell of the present invention. The cells in the composition can be frozen, spray-dried or freeze-dried.

It is additionally possible that the composition comprises a matrix substance. In a preferred embodiment, the matrix substance comprises an excipient selected from sulfoxides and amides e. g. DMSO; alcohols and derivatives e. g. glycerol, glycol, ethylene glycol; sugars e. g. glucose, galactose, lactose, sucrose, trehalose, dextran, erythriol, mannitol, inositol, sorbitol; amino acids e. g. arginine, aspartic acid, glycine, leucine, phenylalanine, thereonine, trileucine, histidine; cellulose derivatives, e.g. hydroxy propyl methyl cellulose starch, starch derivatives e.g., hydroxyethylstarch; organic osmolites e. g. ectoine, 5-hydroxyectoine, taurine, TMAO, gylcine betaine, GPC choline-O-sulphate, ß-DMSP; antioxidants e. g. ascorbic acid, glutathione, α-tocopherol, arbutin, (−)-epigallocatechingallate, chelating agents e. g. EGTA, EDTA, proteins e. g. serum albumin, peptones; other polymers e. g. polyethylene glycol, polyvinyl pyrrolidone, ficoll, poloxameres, carbopol, chitosan.

In a further embodiment, the composition, either in addition to the matrix or the composition without the matrix, comprises a suitable carrier. A suitable carrier or carrier material according to the present invention may be any biocompatible matrix or membrane, for example in form of a wound dressing or gauze including for example films, such as polyurethane films, hydrocarbons, such as petroleum jelly, hydrocolloids, hydrogels, hydrophilic or hydrophobic foams, and calcium alginates, and any solvents, dispersion media, coatings, isotonic solutions and the like, respectively compatible with biologics administration, for example water, saline, ringer's solutions, dextrose solution, and 5% human serum albumin, liposomes and non-aqueous vehicles such as fixed oils known in the art, as well as additives such as absorption delaying agents. The carrier or carrier material may be partially or totally colonized, covered or mixed with the aforementioned cells. In a preferred embodiment of the present invention, the carrier is a wound dressing, preferably a jelly gauze, more preferably petroleum jelly gauze.

Depending on the carrier or carrier material mentioned above, the composition of the present invention is in form of a (wound) dressing, a paste, a spray, or an ointment. Moreover, a product according to the invention is administered or applied topically, subcutaneously or via inhalation In a preferred embodiment, this carrier is a wound dressing, a paste, a spray or an ointment, preferably the carrier is a wound dressing.

Depending on the nature of the wound or inflammatory condition, the composition of the present invention may further comprise a substance selected from the group consisting of antimicrobial agents, antidiabetic agents, mRNA or miRNA antagonists against mRNAs or miRNAs over expressed in chronic, non-healing wounds, growth factors like for example PDGF, FGF-2, VEGF-A, -B, -C, -D, GM-CSF, SDF-1alpha, IL1-beta or peptides derived from these growth factors, inhibitors of enzymes being involved in cortisol synthesis, particularly CYP11B1 overexpressed in chronic wounds as well as of Prolyl-4-hydroxylase, elastase, GSK3ß phosphorylation and Cx43.

Preferably, the antimicrobial agent is an antibiotic and/or an antifungal agent, preferably wherein the antibiotic is selected from the group consisting of: cephalosporines, particularly Cefazolin, Cefoxitin, Cefofetan; macrolides, particularly erythromycin; sulphonamides particularly Mafenide; penicillins, chlorhexidine, silver sulfadiazine, silver nitrate and silver derived formulations, and/or wherein the antidiabetic agent is a DDP-4 inhibitor.

The present invention further refers to the cell of the present invention, or the composition of the present invention, for use in a therapeutic and/or prophylactic treatment. Generally, useful therapeutic and/or prophylactic treatments are those medical situations or diseases where SASP factors are known to be helpful or effective. The cell or the composition can for instance be used in tissue regeneration, and/or in the treatment of wounds. The Wounds can be chronic or acute wounds.

For example, the wounds can be wounds of the skin, preferably wherein the wounds were treated by debridement before treatment with the cell or composition and/or wherein the wounds were generated by split skin grafting and/or plastic surgery.

The wounds can also be burns, particularly a burn selected from the group consisting of superficial and/or deep partial thickness burn, second or third degree burn, sun burn; and/or wherein the wound is a chronic wound healing disorder selected from the group consisting of ulcers, pressure sores, diabetic foot syndrome.

If the wound is an ulcer, the ulcer can be selected from the group consisting of ischemic, arterial, venous, neurotrophic, vasculitis, hypertensive and Pyoderma Gangrenosum, decubitus ulcers. It is also possible that the cell or composition of the present invention is used for treating abrasions, any trauma, radiotherapy lesions, all types of skin loss.

It is also possible that the cell or composition of the present invention is used for treating an inflammatory condition of skin and subcutaneous tissue, wherein the inflammatory condition is preferably selected from the group consisting of: Erythematosquamous dermatosis, Atopic dermatitis and related conditions, Contact dermatitis and other eczema, Dermatitis due to substances taken internally, Bullous dermatoses, Erythematous conditions, Psoriasis and similar disorders, Lichen, Pruritus and related conditions, and other diseases of skin and subcutaneous tissue like Actinic keratosis, Alopecia and Acne.

The present invention further refers to a cell that has been modified by treatment with a suitable amount of an exogenous secretion inhibitor, or composition comprising said modified cell or being derived from said modified cell or its follow-up products, for use in a therapeutic and/or prophylactic treatment as disclosed elsewhere herein. The exogenous secretion inhibitor is an exogenous secretion inhibitor as disclosed elsewhere herein, the modified (treated) cell is a cell as disclosed elsewhere herein, and the composition is a composition as disclosed elsewhere herein. A “suitable” amount of exogenous secretion inhibitor is an amount that is sufficient to significantly reduce, preferably inhibit, the secretion of SASP factor(s) from the modified (treated) cell. As disclosed elsewhere herein, the secretion of the SASP factor(s) is significantly reduced, preferably inhibited, due to the effect the secretion inhibitor exerts on the cellular structure and/or cellular function of the treated cell. The treated cell, as a result of the treatment with the exogenous secretion inhibitor, exhibits a cellular structure or cellular function that is different to the cellular structure or cellular function without the respective treatment, exhibits reduced ability to secrete the at least one SASP-factor, and is intracellularly enriched in the at least one SASP-factor.

The present invention further refers to a method for producing a cell that is intracellularly enriched in at least one endogenously produced SASP factor, comprising the following steps:
(a) providing a cell that is capable of producing one or more SASP factors;
(b) incubating the cell of (i) under suitable conditions with an exogenous secretion inhibitor in an amount that is sufficient to reduce, preferably inhibit, the secretion of the at least one SASP-factor;
(c) obtaining the cell of (ii) that is intracellularly enriched in at least one endogenously produced SASP factor.

With regard to the cell, the SASP factor, and the exogenous secretion inhibitor, reference is made to the disclosure elsewhere herein.

Due to the incubation of the cell of (i) under suitable conditions with an exogenous secretion inhibitor in an amount that is sufficient to reduce, preferably inhibit, the secretion of the at least one SASP-factor, the obtained cell exhibits a cellular structure or cellular function that is different to the cellular structure or cellular function without the respective treatment, exhibits reduced ability to secrete the at least one SASP-factor, and is intracellularly enriched in the at least one SASP-factor.

The following examples illustrate preferred embodiments of the present invention.

### EXAMPLES

### Example 1. Blocking Protein Section of Primary Human Keratinocytes

Y27632 is a commonly used cell-permeable, highly potent, competitive inhibitor of both Rho-associated protein kinases ROCK1 and ROCK2 which suppresses the shedding of extracellular vesicles resulting in a reduced secretion of growth factors associated with the vesicles [54].

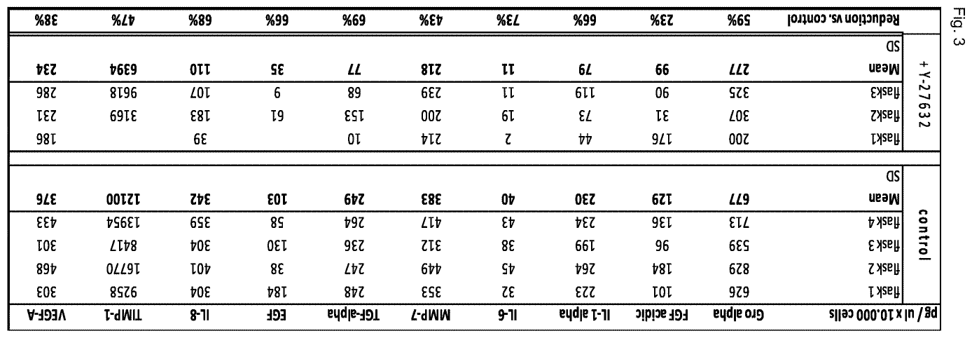

Human primary non-senescent keratinocytes isolated from adult skin and having the capacity of producing at least one SASP factor were cultured in the absence (control) or presence of 10 μM Y-27632 and the concentrations of 10 different growth factors secreted to the medium were determined as described in FIG. 3.

Under the conditions used for testing a significant reduction of the secretion for all growth factors or SASP factors analyzed was observed, ranging from 23% up to 73% depending on the factor analyzed.

### Example 2. Wound Healing Efficacy of Keratinocytes after Blocking Protein Secretion and Freeze Drying

Formation of new dermis is a hallmark of wound healing required to fill up the wound bed with new tissue that was lost during injury. A porcine dermatome model was used to compare the efficacy of formulations composed of primary human keratinocytes after cultivation in the absence (control) or presence of the rock inhibitor Y27632, preserved by freeze drying, to a placebo formulation (freeze drying matrix without cells) and “epifast” (trade name), a keratinocyte-based dressing approved and used to effectively treat burns in Mexico (positive control).

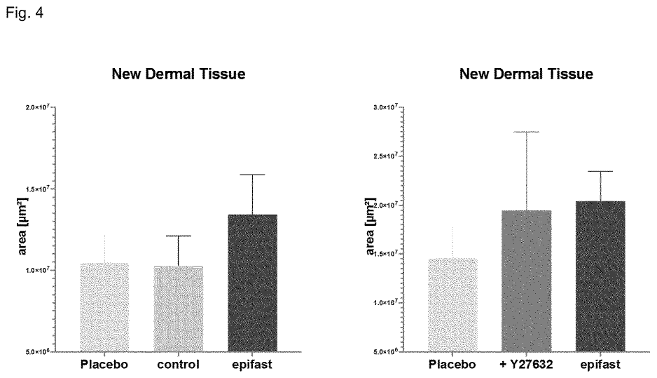

Under anesthesia and analgesia formulations or placebo samples were placed on 3×3 cm sized dermatome wounds of 1.2 mm depth arranged on the flank of three domestic pigs (Sus domesticus) with 6 sites/applications per animal and group. After 7 days the animals were sacrificed following photo documentation and tissue/wound dressing sampling for further morphometric analysis as shown in FIG. 4.

The obtained data show that cells grown in the absence of Y27632 and freeze-drying show no additional effect compared to the placebo and 24% lower compared to the positive control epifast. In contrast to that cells with a decreased secretion after growth in the presence of 10 μM Y27632 and freeze drying show a significant efficacy in formation of new dermis similar to the positive control.

### Applicability

The Examples of the present invention demonstrate that cells, basically having the capacity of producing therapeutically useful factors such as SASP factors, only by means of the present invention adopt a significantly improved status of cells—particularly by adopting a status of being enriched or loaded or even super-loaded with the therapeutically useful factors such as SASP factors—which then can be beneficially used therapeutically. Based on the significant therapeutic findings for tissue regeneration and specifically e.g. to wound healing, the present invention plausibly can be applied also for further therapeutic and/or prophylactic treatments in which the correspondingly enriched or loaded therapeutically active substance such as SASP factors plays a role.

### REFERENCE LIST

1. Hansbrough, J. F., et al., Composite grafts of human keratinocytes grown on a polyglactin mesh-cultured fibroblast dermal substitute function as a bilayer skin replacement in full-thickness wounds on athymic mice. J Burn Care Rehabil, 1993. 14(5): p. 485-94.
2. Kawai, K., et al., Rejection of cultured keratinocyte allografts in presensitized mice. Transplantation, 1993. 56(2): p. 265-9.
3. Rockinger, U., M. Funk, and G. Winter, Current Approaches of Preservation of Cells During (freeze-) Drying. J Pharm Sci, 2021. 110(8): p. 2873-2893.
4. Rockinger, U., et al., DMSO as new, counterintuitive excipient for freeze-drying human keratinocytes. Eur J Pharm Sci, 2021. 160: p. 105746.
5. Petrova, N. V., et al., Small molecule compounds that induce cellular senescence. Aging Cell, 2016. 15(6): p. 999-1017.
6. Serrano, M., Senescence helps regeneration. Dev Cell, 2014. 31(6): p. 671-2.
7. Kumari, R. and P. Jat, Mechanisms of Cellular Senescence: Cell Cycle Arrest and Senescence Associated Secretory Phenotype. Front Cell Dev Biol, 2021. 9: p. 645593.
8. Luesch, H. and V. O. Paavilainen, Natural products as modulators of eukaryotic protein secretion. Nat Prod Rep, 2020. 37(5): p. 717-736.
9. Pawlikowski, J. S., P. D. Adams, and D. M. Nelson, Senescence at a glance. J Cell Sci, 2013. 126(Pt 18): p. 4061-7.
10. Ben-Porath, I. and R. A. Weinberg, When cells get stressed: an integrative view of cellular senescence. J Clin Invest, 2004. 113(1): p. 8-13.
11. Ben-Porath, I. and R. A. Weinberg, The signals and pathways activating cellular senescence. Int J Biochem Cell Biol, 2005. 37(5): p. 961-76.
12. Schmitt, C. A., Senescence, apoptosis and therapy—cutting the lifelines of cancer. Nat Rev Cancer, 2003. 3(4): p. 286-95.
13. Balaban, R. S., S. Nemoto, and T. Finkel, Mitochondria, oxidants, and aging. Cell, 2005. 120(4): p. 483-95.
14. Martin, G. M., Genetic modulation of senescent phenotypes in Homo sapiens. Cell, 2005. 120(4): p. 523-32.
15. Coppé, J. P., et al., Senescence-associated secretory phenotypes reveal cell-nonautonomous functions of oncogenic RAS and the p53 tumor suppressor. PLoS Biol, 2008. 6(12): p. 2853-68.
16. Krtolica, A., et al., Senescent fibroblasts promote epithelial cell growth and tumorigenesis: a link between cancer and aging. Proc Natl Acad Sci USA, 2001. 98(21): p. 12072-7.
17. Coppé, J. P., et al., The senescence-associated secretory phenotype: the dark side of tumor suppression. Annu Rev Pathol, 2010. 5: p. 99-118.
18. Krizhanovsky, V., et al., Senescence of activated stellate cells limits liver fibrosis. Cell, 2008. 134(4): p. 657-67.
19. Sagiv, A., et al., Granule exocytosis mediates immune surveillance of senescent cells. Oncogene, 2013. 32(15): p. 1971-7.
20. Blagosklonny, M. V., Cell cycle arrest is not senescence. Aging, 2011. 3(2): p. 94-101.
21. Coppé, J. P., et al., A role for fibroblasts in mediating the effects of tobacco-induced epithelial cell growth and invasion. Mol Cancer Res, 2008. 6(7): p. 1085-98.
22. Lewis, D. A., et al., UVB-induced senescence in human keratinocytes requires a functional insulin-like growth factor-1 receptor and p53. Mol Biol Cell, 2008. 19(4): p. 1346-53.
23. Volotskova, O., et al., Targeting the cancer cell cycle by cold atmospheric plasma. Sci Rep, 2012. 2: p. 636.
24. Tsai, K. K. C., et al., Cellular Mechanisms for Low-Dose Ionizing Radiation-Induced Perturbation of the Breast Tissue Microenvironment. Cancer Research, 2005. 65(15): p. 6734-6744.
25. Tsai, K. K., et al., Cellular mechanisms for low-dose ionizing radiation-induced perturbation of the breast tissue microenvironment. Cancer Res, 2005. 65(15): p. 6734-44.
26. Papadopoulou, A. and D. Kletsas, Human lung fibroblasts prematurely senescent after exposure to ionizing radiation enhance the growth of malignant lung epithelial cells in vitro and in vivo. Int J Oncol, 2011. 39(4): p. 989-99.
27. Alessio, N., et al., Low dose radiation induced senescence of human mesenchymal stromal cells and impaired the autophagy process. Oncotarget, 2015. 6(10): p. 8155-66.
28. Dierick, J.-F., et al., Identification of 30 protein species involved in replicative senescence and stress-induced premature senescence. FEBS Letters, 2002. 531(3): p. 499-504.
29. Pascal, T., et al., Comparison of replicative senescence and stress-induced premature senescence combining differential display and low-density DNA arrays. FEBS Lett, 2005. 579(17): p. 3651-9.
30. Ewald, J. A., et al., A high-throughput method to identify novel senescence-inducing compounds. J Biomol Screen, 2009. 14(7): p. 853-8.
31. Van Puyenbroeck, V. and K. Vermeire, Inhibitors of protein translocation across membranes of the secretory pathway: novel antimicrobial and anticancer agents. Cell Mol Life Sci, 2018. 75(9): p. 1541-1558.
32. Lindner, P., et al., Cell death induced by the ER stressor thapsigargin involves death receptor 5, a non-autophagic function of MAP1LC3B, and distinct contributions from unfolded protein response components. Cell Commun Signal, 2020. 18(1): p. 12.
33. Lee, A. J., et al., RAS induced senescence of skin keratinocytes is mediated through Rho-associated protein kinase (ROCK). Mol Carcinog, 2021.
34. Chapman, S., et al., The effect of Rho kinase inhibition on long-term keratinocyte proliferation is rapid and conditional. Stem Cell Res Ther, 2014. 5(2): p. 60.
35. D'Souza-Schorey, C. and P. Chavrier, ARF proteins: roles in membrane traffic and beyond. Nat Rev Mol Cell Biol, 2006. 7(5): p. 347-58.
36. Nabors, L. B., B. Surboeck, and W. Grisold, Complications from pharmacotherapy. Handb Clin Neurol, 2016. 134: p. 235-50.
37. Bates, D. and A. Eastman, Microtubule destabilising agents: far more than just antimitotic anticancer drugs. Br J Clin Pharmacol, 2017. 83(2): p. 255-268.
38. Khwaja, S., et al., Microtubule associated proteins as targets for anticancer drug development. Bioorg Chem, 2021. 116: p. 105320.
39. Gronberg, A., et al., Antioxidants protect keratinocytes against M. ulcerans mycolactone cytotoxicity. PLoS One, 2010. 5(11): p. e13839.
40. Ogbechi, J., et al., Inhibition of Sec61-dependent translocation by mycolactone uncouples the integrated stress response from ER stress, driving cytotoxicity via translational activation of ATF4. Cell Death Dis, 2018. 9(3): p. 397.
41. Westendorf, C., et al., Inhibition of biosynthesis of human endothelin B receptor by the cyclodepsipeptide cotransin. J Biol Chem, 2011. 286(41): p. 35588-35600.
42. Paatero, A. O., et al., Apratoxin Kills Cells by Direct Blockade of the Sec61 Protein Translocation Channel. Cell Chem Biol, 2016. 23(5): p. 561-566.
43. Junne, T., et al., Decatransin, a new natural product inhibiting protein translocation at the Sec61/SecYEG translocon. J Cell Sci, 2015. 128(6): p. 1217-29.
44. Tranter, D., et al., Coibamide A Targets Sec61 to Prevent Biogenesis of Secretory and Membrane Proteins. ACS Chem Biol, 2020. 15(8): p. 2125-2136.
45. Roboti, P., et al., Ipomoeassin-F disrupts multiple aspects of secretory protein biogenesis. Sci Rep, 2021. 11(1): p. 11562.
46. Zhao, L., et al., Identification of pharmacological inhibitors of conventional protein secretion. Sci Rep, 2018. 8(1): p. 14966.
47. Shi, B. and R. R. Isseroff, Thapsigargin induces phosphorylation of the 27-kDa heat shock protein in human keratinocytes. J Invest Dermatol, 1996. 107(5): p. 749-54.
48. Quynh Doan, N. T. and S. B. Christensen, Thapsigargin, Origin, Chemistry, Structure-Activity Relationships and Prodrug Development. Curr Pharm Des, 2015. 21(38): p. 5501-17.
49. Navarrete, C., et al., Basiliolides, a class of tetracyclic C19 dilactones from Thapsia garganica, release Ca(2+) from the endoplasmic reticulum and regulate the activity of the transcription factors nuclear factor of activated T cells, nuclear factor-kappaB, and activator protein 1 in T lymphocytes. J Pharmacol Exp Ther, 2006. 319(1): p. 422-30.
50. McGuire, J. and N. Fedarko, Keratinocytes form multi-cell clusters in the absence of cell division or collagen synthesis; tunicamycin interferes with cell cluster formation. Curr Probl Dermatol, 1983. 11: p. 83-96.
51. Jin, S. P. and J. H. Chung, Inhibition of N-glycosylation by tunicamycin attenuates cell-cell adhesion via impaired desmosome formation in normal human epidermal keratinocytes. Biosci Rep, 2018. 38(6).
52. Estoppey, D., et al., The Natural Product Cavinafungin Selectively Interferes with Zika and Dengue Virus Replication by Inhibition of the Host Signal Peptidase. Cell Rep, 2017. 19(3): p. 451-460.
53. Nam, Y. J. and C. S. Lee, Brefeldin A reduces tumor necrosis factor-alpha-stimulated production of inflammatory mediators by suppressing the Akt, mTOR, and NF-kappaB pathways in human keratinocytes. Naunyn Schmiedebergs Arch Pharmacol, 2016. 389(9): p. 951-60.
54. Catalano, M. and L. O'Driscoll, Inhibiting extracellular vesicles formation and release: a review of EV inhibitors. J Extracell Vesicles, 2020. 9(1): p. 1703244.
55. Zimmerman, U. J. P., M. Wang, and L. Liu, Inhibition of secretion from isolated rat alveolar epithelial type II cells by the cell permeant calpain inhibitor II (N-acetyl-leucyl-leucyl-methioninal). Cell Calcium, 1995. 18(1): p. 1-8.
56. Liu, Y. M., et al., Tubulin inhibitors: a patent review. Expert Opin Ther Pat, 2014. 24(1): p. 69-88.
57. Mundhara, N., A. Majumder, and D. Panda, Methyl-beta-cyclodextrin, an actin depolymerizer augments the antiproliferative potential of microtubule-targeting agents. Sci Rep, 2019. 9(1): p. 7638.
58. Owellen, R. J., et al., Inhibition of tubulin-microtubule polymerization by drugs of the Vinca alkaloid class. Cancer Res, 1976. 36(4): p. 1499-502.
59. Jordan, M. A. and L. Wilson, Microtubules as a target for anticancer drugs. Nature Reviews Cancer, 2004. 4(4): p. 253-265.
60. Diez, J. C., et al., Reversible inhibition of microtubules and cell growth by the bicyclic colchicine analogue MTC. Cell Motil Cytoskeleton, 1987. 7(2): p. 178-86.
61. Chou, T. C., et al., Desoxyepothilone B is curative against human tumor xenografts that are refractory to paclitaxel. Proc Natl Acad Sci USA, 1998. 95(26): p. 15798-802.
62. Bajaj, M. and M. Srayko, Laulimalide induces dose-dependent modulation of microtubule behaviour in the C. elegans embryo. PLoS One, 2013. 8(8): p. e71889.
63. Havercroft, J. C., R. A. Quinlan, and K. Gull, Binding of parbendazole to tubulin and its influence on microtubules in tissue-culture cells as revealed by immunofluorescence microscopy. J Cell Sci, 1981. 49: p. 195-204.
64. Jiang, C. and Q. You, Kinesin spindle protein inhibitors in cancer: a patent review (2008-present). Expert Opin Ther Pat, 2013. 23(12): p. 1547-60.
65. Voultsiadou, A. and V. Sarli, Recent advances of kinesin motor inhibitors and their clinical progress. Rev Recent Clin Trials, 2011. 6(3): p. 271-7.
66. Cheng, Y., et al., Therapeutic targeting of autophagy in disease: biology and pharmacology. Pharmacol Rev, 2013. 65(4): p. 1162-97.
67. Wang, R., B. Sunchu, and V. I. Perez, Rapamycin and the inhibition of the secretory phenotype. Exp Gerontol, 2017. 94: p. 89-92.
68. Schreiber, K. H., et al., A novel rapamycin analog is highly selective for mTORC1 in vivo. Nat Commun, 2019. 10(1): p. 3194.
69. Atkin, J., et al., Torin1-mediated TOR kinase inhibition reduces Wee1 levels and advances mitotic commitment in fission yeast and HeLa cells. J Cell Sci, 2014. 127(Pt 6): p. 1346-56.
70. Gao, L., et al., An investigation of the metabolic activity, isozyme contribution, species differences and potential drug-drug interactions of P1-103, and the identification of efflux transporters for PI-103-O-glucuronide in HeLa1A9 cells. RSC Adv, 2020. 10(16): p. 9610-9622.
71. Dettmer, S., et al., Proteasome inhibition correlates with intracellular bortezomib concentrations but not with antiproliferative effects after bolus treatment in myeloma cell lines. Naunyn Schmiedebergs Arch Pharmacol, 2016. 389(10): p. 1091-101.
72. Catley, L., et al., NVP-LAQ824 is a potent novel histone deacetylase inhibitor with significant activity against multiple myeloma. Blood, 2003. 102(7): p. 2615-22.
73. Song, J. H., et al., The BH3 mimetic ABT-737 induces cancer cell senescence. Cancer Res, 2011. 71(2): p. 506-15.
74. Criollo, A., et al., Regulation of autophagy by the inositol trisphosphate receptor. Cell Death Differ, 2007. 14(5): p. 1029-39.
75. Ahumada-Castro, U., et al., MTOR-independent autophagy induced by interrupted endoplasmic reticulum-mitochondrial Ca(2+) communication: a dead end in cancer cells. Autophagy, 2019. 15(2): p. 358-361.
76. Ma, T., et al., Low-dose metformin targets the lysosomal AMPK pathway through PEN2. Nature, 2022. 603(7899): p. 159-165.
77. Aasland, D., et al., Temozolomide Induces Senescence and Repression of DNA Repair Pathways in Glioblastoma Cells via Activation of ATR-CHK1, p21, and NF-kappaB. Cancer Res, 2019. 79(1): p. 99-113.
78. Chen, K. Y., et al., Resveratrol induced premature senescence and inhibited epithelial-mesenchymal transition of cancer cells via induction of tumor suppressor Rad9. PLoS One, 2019. 14(7): p. e0219317.
79. Marshall, A. M., et al., Serotonin transport and metabolism in the mammary gland modulates secretory activation and involution. J Clin Endocrinol Metab, 2010. 95(2): p. 837-46.
80. Lowes, S. and N. L. Simmons, Multiple pathways for fluoroquinolone secretion by human intestinal epithelial (Caco-2) cells. Br J Pharmacol, 2002. 135(5): p. 1263-75.
81. Niessner, H., et al., The farnesyl transferase inhibitor lonafarnib inhibits mTOR signaling and enforces sorafenib-induced apoptosis in melanoma cells. J Invest Dermatol, 2011. 131(2): p. 468-79.
82. Ahn, J. H., S. K. Ahn, and M. Lee, The role of autophagy in cytotoxicity induced by new oncogenic B-Raf inhibitor UI-152 in v-Ha-ras transformed fibroblasts. Biochem Biophys Res Commun, 2012. 417(2): p. 857-63.
83. Yang, S., et al., Pancreatic cancers require autophagy for tumor growth. Genes Dev, 2011. 25(7): p. 717-29.
84. Morozkin, E. S., et al., The effect of protein transport inhibitors on the production of extracellular DNA. Ann NY Acad Sci, 2008. 1137: p. 31-5.
85. Billinger, E., et al., Inhibition properties of free and conjugated leupeptin analogues. FEBS Open Bio, 2020. 10(12): p. 2605-2615.
86. Khandke, L., et al., Cyclosporine in psoriasis treatment. Inhibition of keratinocyte cell-cycle progression in G1 independent of effects on transforming growth factor alpha/epidermal growth factor receptor pathways. Arch Dermatol, 1991. 127(8): p. 1172-9.
87. Eichelberg, D. and A. Fuchs, Calmodulin-antagonism inhibits human keratinocyte proliferation. Arch Dermatol Res, 1988. 280(5): p. 323-4.
88. Clark, E. A., et al., Laulimalide and synthetic laulimalide analogues are synergistic with paclitaxel and 2-methoxyestradiol. Mol Pharm, 2006. 3(4): p. 457-67.

1. A cell having the capacity of producing at least one SASP factor, obtainable or obtained by treating the cell with an amount of an exogenous secretion inhibitor that is sufficient to reduce the secretion of the at least one SASP-factor.

2. The cell according to claim 1, wherein the cell, as a result of the treatment with the exogenous secretion inhibitor, exhibits the following capacities (i) to (iii):
(i) a cellular structure or cellular function that is different to the cellular structure or cellular function without the respective treatment;
and
(ii) reduced ability to secrete the at least one SASP-factor; and/or
(iii) is intracellularly enriched in the at least one SASP-factor.

3. The cell according to claim 1, wherein the at least one SASP factor is selected from the group consisting of IL-1α, IL-10, IL-6, IL-7, IL-8, IL-10, IL-13, IL-15, IL-18, MCP1, MCP2, MCP4, MIF, MIP-1a, MIP-3a, HCC-4, Eotaxin-3, TECK, ENA-78, I-309, I-TAC, GROα, GROβ, GROγ, VEGF, EGF, HGF, HBEGF, FGF, NGF, Amphiregulin, Angiogenin, APOJ, CAV1, OSTEO, Epiregulin, Heregulin, SCF, SDF-1 alpha, PIGF, IGF-1, IGFBP-1, -2, -3, -4, -5, -6, -7, GM-CSF, PDGF-BB, TGF-α, TGF-β1, TGF-β2, TGF-β3, TRAIL-R3, Fas, OPG, SGP130, EGF-R uPAR, sTNFRI, sTNFRIII, MMP1, MMP2, MMP3, MMP7, MMP9, MMP10, MMP12, MMP13, MMP14, TIMP1, TIMP2, PAI1, PAI2, Park7 DJ-1, uPA/Urokinase, SLPI, SPP1, Syndecan 1, -4, Tenascin C, Endothelin, Collagens, Fibronectins, Laminins, ICAM1, and ICAM3.

4. The cell according to claim 2, wherein the cellular structure or cellular function that is different to the respective cellular structure or cellular function without the respective treatment is one or more selected from the group consisting of:
(i-i) function of protein insertion into the endoplasmic reticulum (ER);
(i-ii) function of protein maturation;
(i-iii) function of protein trafficking and/or vesicle formation;
(i-iv) function of microtubules; and
(i-v) function of kinesin;
(i-vi) function of autophagy

5. The cell according to claim 1, wherein the exogenous secretion inhibitor is selected from the group consisting of any of (i) to (vi):
(i) Group I: exogenous secretion inhibitor that affects the function of import into the endoplasmic reticulum (ER);
(ii) Group II: exogenous secretion inhibitor that affects the function of protein maturation;
(iii) Group III: exogenous secretion inhibitor that affects the function of protein trafficking and/or vesicle formation;
(iv) Group IV: exogenous secretion inhibitor that affects the function of microtubules;
(v) Group V: exogenous secretion inhibitor that affects the function of kinesin; and
(vi) Group VI: exogenous secretion inhibitor that affects the function of autophagy.

6. The cell according to claim 5, wherein
(i) the exogenous secretion inhibitor from Group I is selected from the group of compounds that affect endoplasmic reticulum (ER) import (group I);
(ii) the exogenous secretion inhibitor from Group II is selected from the group of compounds that affect maturation of proteins (group II);
(iii) the exogenous secretion inhibitor from Group III is selected from the group of compounds that affect protein trafficking and/or vesicle formation (group III);
(iv) the exogenous secretion inhibitor from Group IV is selected from the group of compounds that affect tubulin (group IV);
(v) the exogenous secretion inhibitor from Group V is selected from the group of compounds that affect the function of kinesin;
(vi) the exogenous secretion inhibitor from Group VI is selected from the group of compounds that affect autophagys.

7. The cell according to claim 1, wherein the cell is selected from the group consisting of keratinocytes, fibroblasts, epithelial cells, melanocytes, endothelial cells, corneal epithelial cells, pericytes, monocytes, lymphocytes, thrombocytes, mast cells, adipocytes, muscle cells, neurons, osteocytes, osteoblasts, chondrocytes, mesenchymal stem cells, and adult or embryonic stem cells.

8. The cell according to claim 1, wherein the cell is of a senescent cell type.

9. The cell according to claim 8, wherein the senescent cell type derives from cells selected from the group consisting of keratinocytes, fibroblasts, epithelial cells, melanocytes, endothelial cells, corneal epithelial cells, pericytes, monocytes, lymphocytes, thrombocytes, mast cells, adipocytes, muscle cells, neurons, osteocytes, osteoblasts, chondrocytes, mesenchymal stem cells, and adult or embryonic stem cells.

10. A cell being intracellularly enriched in at least one SASP-factor selected from the group consisting of IL-1 alpha, IL-6, IL8, TGF-alpha, GRO alpha, VEGF, EGF, FGF acid, MMP7, and TIMP1, wherein the intracellular concentration, calculated as pg/mg protein extract, of said one or more SASP factor, if present, is defined as follows:

| | intracellular |
|---|---|
| | (pg/mg protein |
| | extract) |
| IL-1 alpha | more than 80 |
| IL-6 | more than 2 |
| TGF- alpha | more than 20 |
| IL8 | more than 20 |
| GRO alpha | more than 20 |
| VEGF | more than 20 |
| EGF | more than 10 |
| FGF acidic | more than 100 |
| MMP7 | more than 200 |
| TIMP1 | |
| TIMP1 | more than 200 |

11. The cell according to claim 1, wherein the cell is a frozen, spray-dried or freeze-dried cell, or the cell is present in or transferred into a frozen, dried, spray-dried or freeze-dried composition.

12-29. (canceled)

30. A method for producing a cell that is intracellularly enriched in at least one endogenously produced SASP factor, comprising the following steps:
(a) providing a cell that is capable of producing one or more SASP factors;
(b) incubating the cell of (i) under suitable conditions with an exogenous secretion inhibitor in an amount that is sufficient to reduce the secretion of the at least one SASP-factor;
(c) obtaining the cell of (ii) that is intracellularly enriched in at least one endogenously produced SASP factor.

31. The method according to claim 30, wherein the obtained cell in step (c)
(i) exhibits a cellular structure or cellular function that is different to the cellular structure or cellular function without the respective treatment;
and
(ii) exhibits reduced ability to secrete the at least one SASP-factor; and/or
(iii) is intracellularly enriched in the at least one SASP-factor.

32. The cell according to claim 6, wherein the exogenous secretion inhibitor from Group III is a rock-inhibitor.

33. The cell as defined in claim 1, which is comprised in a composition together with a matrix substance or an excipient selected from sulfoxides and amides; alcohols and derivatives; sugars; amino acids; cellulose derivatives; organic osmolites; antioxidants; chelating agents; proteins; and other polymeric substances selected from the groups of polyethylene glycol, polyvinyl pyrrolidone, ficoll, poloxameres, carbopol, and chitosan.

34. The cell according to claim 33, wherein the composition further comprises, or is applied to, a carrier selected from the group consisting of a wound dressing, a paste, a spray or an ointment.

35. The cell according to claim 33, wherein the composition additionally contains a functional substance selected from the group of: antimicrobial agents, antidiabetic agents, mRNA or miRNA antagonists against mRNAs or miRNAs over expressed in chronic, non-healing wounds, growth factors, inhibitors of enzymes being involved in cortisol synthesis, as well as of Prolyl-4-hydroxylase, elastase, GSK3ß phosphorylation and Cx43.

36. A method for therapeutic and/or prophylactic treatment, comprising in a first step providing a cell according to claim 1, and in a second step administering said cell to a person in need thereof, wherein the treatment is tissue regeneration, treatment of wounds, or treatment of an inflammatory condition of skin and subcutaneous tissue.

37. The method for therapeutic treatment according to claim 36, wherein the wounds are wounds of the skin.

38. The method for therapeutic treatment according to claim 36, wherein the wounds are burns selected from the group consisting of: superficial and/or deep partial thickness burn, second or third degree burn, sun burn; and/or wherein the wound is a chronic wound healing disorder selected from the group consisting of ulcers, pressure sores, diabetic foot syndrome.

39. A method for therapeutic and/or prophylactic treatment, comprising in a first step providing a cell according to claim 33, and in a second step administering said cell to a person in need thereof, wherein the treatment is tissue regeneration, treatment of wounds, or treatment of an inflammatory condition of skin and subcutaneous tissue.

40. The method for therapeutic treatment according to claim 39, wherein the wounds are wounds of the skin.

41. The method for therapeutic treatment according to claim 39, wherein the wounds are burns selected from the group consisting of: superficial and/or deep partial thickness burn, second or third degree burn, sun burn; and/or wherein the wound is a chronic wound healing disorder selected from the group consisting of ulcers, pressure sores, diabetic foot syndrome.

42. A method for producing a cell that is intracellularly enriched in at least one endogenously produced SASP factor, comprising the following steps:
(a) providing a cell that is capable of producing one or more SASP factors;
(b) incubating the cell of (i) under suitable conditions with an exogenous secretion inhibitor in an amount that is sufficient to reduce the secretion of the at least one SASP-factor;
(c) obtaining the cell of (ii) that is intracellularly enriched in at least one endogenously produced SASP factor.
